SEQUENCE LISTING (2 sequences)

SEQ ID NO: 1 - protein, 21 aa, Artificial Sequence
  Synthetic peptide.
YEDSYEDISAYLLSKNNAIPR

SEQ ID NO: 2 - protein, 25 aa, Artificial Sequence
  Synthetic peptide.
DALRIQMEERFMASNPSKVSYEPIT

(19) United States
(12) Patent Application Publication
(10) Pub. No.: US 2004/0253576 A1
(43) Pub. Date: Dec. 16, 2004

(54) Modulating cooperative activity of dopamine D1 and D2 receptors to mitigate substance abuse

(75) Inventors: Antonello Bonci, San Francisco, CA (US); F. Woodward Hopf, Berkeley, CA (US); Ivan Diamond, Berkeley, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/818,709

(22) Filed: Apr. 5, 2004

Related U.S. Application Data
(60) Provisional application No. 60/460,270, filed on Apr. 4, 2003.

(51) Int. Cl. C12Q 1/00; A61K 31/195

(52) U.S. Cl. 435/4

(57) ABSTRACT

This invention provides to the discovery of the mechanism of a synergistic activity between dopamine D1 and D2 receptors and the exploitation of this mechanism to mitigate one or more symptoms associated with consumption of a substance of abuse. In certain embodiments, this invention provides a method of inhibiting nucleus accumbens spike firing in response to administration of a substance of abuse, where the method involves increasing activity of a slow A-type potassium current (IAS) in cells of the nucleus accumbens.

### CROSS-REFERENCE TO RELATED APPLICATIONS

This application claims priority to and benefit of U.S. Ser. No. 60/460,270, filed Apr. 4, 2003, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the field of neurobiology and substance abuse. In particular this invention pertains to the discovery of the mechanism of a synergistic activity between dopamine D1 and D2 receptors and the exploitation of this mechanism to mitigate one or more symptoms associated with consumption of a substance of abuse.

### BACKGROUND OF THE INVENTION

Dopamine (DA) in the nucleus accumbens (NAcb) has long been considered an important modulator of addiction and goal-directed behaviors (Spanagel and Weiss, 1999). The shell region of the NAcb in particular is implicated in a number of cellular and behavioral phenomena, especially in relation to addictive drugs (Zahm, 1999). Although a diversity of functional effects and ionic targets can be modulated by DA, the exact role of DA receptor activation in the NAcb is only partially understood (Greengard et al., 1999; Nicola et al., 2000). DA receptors are generally grouped into two sub-families, the D₁-like receptors and the D₂-like receptors (Missale et al., 1998). Opposing influences of D₁ and D₂ receptor activation on cAMP-dependent signaling have been reported in many studies (Stoof and Kebabian, 1981, Missale et al., 1998), with D₁ receptors acting through the stimulatory Gₛ-like G_{olf}, and D₂ receptors acting through the inhibitory G_{i/o} proteins.

In contrast, results from a number of behavioral studies suggest a cooperative interaction of D₁ and D₂ receptors in the NAcb (Plaznik et al., 1989; Chu and Kelley, 1992; Wolterink et al., 1993; Phillips et al., 1994; Hodge et al., 1997; Ikemoto et al., 1997; Gong et al., 1999; Koch et al., 2000; Nowend et al., 2001). For example, animals will self-administer D₁ and D₂ agonists directly into the NAcb in combination but not alone (Ikemoto et al., 1997). Further, self-administration of amphetamine (Phillips et al., 1994) and ethanol (Hodge et al., 1997), lever pressing for a conditioned reinforcer (Chu and Kelley, 1992; Wolterink et al., 1993), and evaluating the relative cost of obtaining a reward (Koch et al., 2000; Nowend et al., 2001) all may involve co-activation of D₁ and D₂ receptors in the NAcb. Dose-dependent modulation of firing rate and TTX-independent Fos induction by co-operative D₁/D₂ receptor activation have also been reported (Chiodo and Berger, 1986; Wachtel et al., 1989; Williams and Millar, 1990; Hu and White, 1997; LaHoste et al., 2000).

Despite the implication of D₁/D₂ receptor cooperativity in several behaviors, the specific cellular and biochemical pathways that mediate the interaction between D₁ and D₂ receptors are uncertain.

### SUMMARY OF THE INVENTION

Dopamine in the nucleus accumbens modulates both motivational and addictive behaviors. Dopamine D₁ and D₂ receptors have previously been considered to exert opposite effects at the cellular level. Here, we show that a dopamine-induced enhancement of spike firing in nucleus accumbens neurons in brain slice required both D₁ and D₂ receptors. This enhancement was prevented by inhibitors of PKA or G-protein beta-gamma subunits. Finally, our data suggest that these pathways may increase spike firing by inhibition of a slow A-type potassium current. These results provide evidence for a novel cellular mechanism through which cooperative action of D₁ and D₂ receptors in the nucleus accumbens could mediate dopamine-dependent behaviors.

The discovery of this cooperativity provides new approaches to mitigating the effects associated with chronic consumption and/or withdrawal of a substance of abuse. In addition, the discovery of this mechanism provides new targets to screen for agents suitable in the treatment of substance abuse and/or withdrawal from consumption of a substance of abuse.

Thus, in certain embodiments, this invention provides a method of screening for an agent that modulates self-administration of a substance of abuse. The method typically involves contacting a neural cell with a test agent; and determining whether the test agent agonizes activity of a slow A-type potassium current (IAS), wherein an increase in the activity of the potassium current indicates that said test agent is an agent that is expected to inhibit self-administration of a substance of abuse. In certain embodiments, the determination is by an electrophysiological measurement. In certain embodiments, the neural cell is a cell in a brain tissue, preferably in a brain slice preparation, more preferably a brain slice preparation comprising tissue of the nucleus accumbens. Preferred test agents include, but are not limited to small organic molecules.

This invention also provides a method of inhibiting nucleus accumbens spike firing in response to administration of a substance of abuse (e.g., ethanol, an opiate, a cannabinoid, a stimulant, and nicotine). The method typically involves increasing activity of a slow A-type potassium current (IAS) in cells of the nucleus accumbens. In certain embodiments, the method involves administering a small organic molecule that inhibits activity of the slow A-type potassium current.

Also provided is a method of inhibiting self-administration of a substance of abuse. The method typically involves increasing activity of a slow A-type potassium current (I_{AS}). In certain embodiments, the substance of abuse is alcohol. The method can involve administering a small organic molecule that inhibits activity of the slow A-type potassium current. The method can also involve electrophysiologically inhibiting the slow A-type potassium current.

In still another embodiment, this invention provides a composition for mitigating symptoms of consumption or withdrawal of a substance of abuse. The composition comprises a modulator of a slow A-type potassium current and, optionally, a pharmacologically acceptable excipient.

Definitions

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs.

A “dopamine receptor antagonist” refers to a substance that reduces or blocks activity mediated by a dopamine receptor in response to the cognate ligand of that receptor. Thus, for example, a dopamine receptor antagonist will reduce or eliminate the activity of dopamine mediated by a dopamine receptor and associated pathway(s). The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity.

As used herein, the term “dopamine receptor agonist” means an agent capable of combining with D2 dopamine receptor and capable of stimulating the associated receptor activity. The term dopamine receptor agonist will also include partial dopamine receptor agonists that are capable of partially stimulating D2 activity, i.e., providing a lesser activity than would be obtained with a like concentration of dopamine.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “database” refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrases “an amount [of an agent] sufficient to maintain changes in gene expression” or “an amount [of an agent] sufficient to induce changes in gene expression” refers to the amount of the “agent” sufficient maintain or induce those changes in the subject organism as empirically determined or as extrapolated from an appropriate model system.

### BRIEF DESCRIPTION OF THE DRAWINGS

FIG. 1 Dopamine (75 μM) increased spike firing in MSNs from the NAcb shell. A, Example of applied current pulses and voltage responses. The traces represent sub-threshold current pulses and the first pulse able to elicit spikes. Current pulses eliciting a greater number of spikes are not shown. Vm-rest was −79 mV. B, Input-output relationship showing significant enhancement of spike firing by DA for all current pulses greater than 150 pA in magnitude (p<0.05, paired t-test). Spike rate for each cell was normalized to the number of spikes elicited by a 350 pA current pulse before addition of DA (10.0+/−1.4 spikes). C, Example traces showing reversible increase in spike firing in the presence of DA (250 pA current pulse). Vm-rest of the example traces shown were −83.2 mV, −80.8 mV, and −83.0 mV. C, Time course experiment demonstrating an enhancement of spike firing after application of DA. Data correspond to examples of spike firing shown in B. All data shown in A to D are from perforated patch recordings. E, Compiled data from neurons exposed to DA during whole-cell (open circles) and perforated patch (closed circles) recordings.

FIG. 2 Co-activation of both D₁ and D₂ receptors was required to increase spike firing. A, Examples showing (Al) no effect of a D₁ agonist (SKF82957, 10 μM) or a D₂ agonist (quinpirole, 10 μM) alone on spike rate, but (A2) an increase in spike rate with a combination of 10 μM each of a D₁ (SKF82957) and a D₂ receptor agonist (quinpirole). B, Compiled data from neurons exposed to a D₁ agonist (SKF82957 or SKF81297, 10 μM) or a D₂ agonist (quinpirole, 10 μM) alone or in combination. C, Pre-exposure to a selective D₁ antagonist (SCH23390, 10 μM) or a selective D₂ antagonist (eticlopride, 3 μM) prevented DA-mediated increases in spike firing. DA data are the same whole-cell results shown in FIG. 1 D, to assist in comparison of the effects of DA with and without receptor antagonists. D, Compiled data showing dose-response of spike firing enhancement by dopamine receptor agonists. “Per.” and “Wh.” indicate experiments performed using perforated patch or whole-cell recording. E, Enhancement of synaptically-evoked spike firing by a combination of 3 μM each of SKF81297 and quinpirole. Vm-rest was −81.6 mV before agonists and −82.3 mV after. *(D, E) indicates significant increase in spike firing (p<0.05, paired t-test).

FIG. 3 PKA activation was required for DA-mediated increases in spike firing. A, Application of 100 μM Rp-cAMPS during the plateau DA response reduced spike firing to baseline levels. Only neurons showing a change in spike firing with DA were tested for effects of Rp-cAMPS. B, Forskolin, an activator of the PKA system, led to a similar increase in spike rate as DA, while dideoxyforskolin, the inactive control, had no effect. C, Inhibition of protein phosphatases 1 and 2A via intracellular perfusion with okadaic acid (1 μM) enhanced spike firing and occluded DAergic effects, while the inactive analog norokadaone (1 μM) had no effect alone and did not occlude DA-mediated enhancement of spike firing. In legend, “ok.ac.” indicates intracellular perfusion with okadaic acid, while “norok.” indicates intracellular perfusion with norokadaone. D, Model showing a possible intracellular pathway by which a combination of a D₁ and a D₂ receptor agonist can activate PKA. “AC” indicates adenylyl cyclase. We should note that there may be intervening molecules between PKA and I_{As}, and also that D₁ and D₂ receptors might be on the same or different cells.

FIG. 4 Gβγ was required for the increased spike firing produced by co-application of a D₁ and a D₂ receptor agonist. A, Example traces showing increased spike firing during dialysis with FVIII, the inactive control (A1, 250 pA current pulse), but not when Spβγ, the G_{βγ} inhibitory peptide, was present in the intracellular solution (A2, 250 pA current pulse). For traces in A1, Vm-rest was −78.4, −80.4, and −78.5 mV. For traces in A2, Vm-rest was −79.6, −81.1, and −82.5 mV. B, Time course experiment demonstrating that SPβγ, but not FVIII, prevented the enhancement of spike firing after co-application of a D₁ and a D₂ receptor agonist. C, Forskolin-mediated increases in spike rate were not affected by either FVIII or SPβγ. D, Intracellular perfusion with G_{βγ} subunits enabled enhancement of spike firing by D₁ but not D₂ receptor agonists.

FIG. 5 DAergic agonists enhanced spike firing without altering several electrophysiological parameters. A, Exposure to DA or D₁ and D₂ agonists (10 μM), either alone or in combination, did not significantly change resting membrane potential (Vm-rest, A1) or input resistance (A2) of MSNs. “DA-Per.” and “DA-Wh.” indicate neurons exposed to DA during perforated patch and whole-cell recording, respectively. B, Distribution of percent change in spiking versus change in Vm-rest for cells exposed to DAergic agonists. C, Example trace showing change in 3.2% change in spike firing per mV increase (calculated for the 250 pA pulse). D, Example traces of spike firing before and after exposure to a combination of a D₁ and a D₂ receptor agonist (250 pA current pulse). Inset demonstrates that action potential threshold and width and the relative hyperpolarization peak were unchanged. Vm-rest for the traces shown was −83.7 and −80.1 mV. E, Averaged data for action potential peak (APP), action potential width (APW), and fast afterhyperpolarization (ƒAHP) before (dark column) and after (light column) exposure to a D₁ and a D₂ receptor agonist in combination.

FIG. 6. Inhibition of I_{AS} increases spike firing. A, α-dendrotoxin (0.5 μM) increased spike firing. B, Pre-exposure to α-dendrotoxin occluded the effects of DA. C, 4-AP increased spike firing in a dose-dependent manner. Data for 5, 10, 20, 40, and 60 μM 4-AP were collected from 9, 11, 4, 7, and 6 neurons, respectively. D, Pre-exposure to 60 μM 4-AP occluded the DA-mediated enhancement of spike firing. However, glutamate (200 μM) was able to further increase spike rate significantly. E, Example showing that exposure to 4-AP (10 μM, E1) or DA (75 μM, E2) led to a small but significant reduction in the total potassium current evoked during voltage-clamp experiments in which cells were depolarized from −80 to 0 mV. F, Compiled data showing reduction of total potassium current by DA or 4-AP.

### DETAILED DESCRIPTION

Dopamine in the nucleus accumbens modulates both motivational and addictive behaviors. Dopamine D₁ and D₂ receptors are generally considered to exert opposite effects at the cellular level, but many behavioral studies find an apparent cooperative effect of D₁ and D₂ receptors in the nucleus accumbens. We have discovered that a dopamine-induced enhancement of spike firing in nucleus accumbens neurons in brain slice required both D₁ and D₂ receptors. One intracellular mechanism that we believe underlies cooperativity of D₁ and D₂ receptors is activation of specific subtypes of adenylyl cyclases by G-protein beta-gamma subunits (G_{βγ}) released from the G_{i/o}-linked D₂ receptor in combination with G_{αs}-like subunits from the D₁ receptor. In this regard, dopaminergic enhancement of spike firing was prevented by inhibitors of PKA or G_{βγ}. Further, intracellular perfusion with G_{βγ} enabled D₁ receptor activation to enhance spike firing, while D₂ receptor function was not altered. Finally, our data suggest that these pathways increase spike firing by inhibition of a slow A-type potassium current. These results provide evidence for a novel cellular mechanism through which cooperative action of D₁ and D₂ receptors in the nucleus accumbens can mediate dopamine-dependent behaviors.

These observations can be exploited to provide new approaches to mitigating the effects associated with chronic consumption and/or withdrawal of a substance of abuse. In addition, the discovery of this mechanism provides new targets to screen for agents suitable in the treatment of substance abuse and/or withdrawal from consumption of a substance of abuse.

Thus, in certain embodiments, this invention contemplates screening test agents for the ability to modulate (e.g. increase or decrease) the activity of a slow A-type potassium current in a neural cell. Having identified the relevant current, using the teachings provided herein, screening a test agent for the ability to increase or decrease such a current is routine. Example 1 illustrates screening of various test agents for such activity either directly or for their effect on spike activity in NA.

Agonists (upregulators) of the slow A-type potassium current are expected to decrease NA spike firing and to reduce the drive of a subject (human or non-human mammal) to self-administer one or more substances of abuse.

### EXAMPLES

The following examples are offered to illustrate, but not to limit the claimed invention.

### Example 1

### Cooperative Activation of Dopamine D₁ and D₂ Receptors Increases Spike Firing of Nucleus Accumbens Neurons via G-protein βγ Subunits

Dopamine in the nucleus accumbens modulates both motivational and addictive behaviors. Dopamine D₁ and D₂ receptors are generally considered to exert opposite effects at the cellular level, but many behavioral studies find an apparent cooperative effect of D₁ and D₂ receptors in the nucleus accumbens. In this example, we show that a dopamine-induced enhancement of spike firing in nucleus accumbens neurons in brain slice required both D₁ and D₂ receptors. One intracellular mechanism that might underlie cooperativity of D₁ and D₂ receptors is activation of specific subtypes of adenylyl cyclases by G-protein beta-gamma subunits (G_{βγ}) released from the G_{i/o}-linked D₂ receptor in combination with G_{αs}-like subunits from the D₁ receptor. In this regard, dopaminergic enhancement of spike firing was prevented by inhibitors of PKA or G_{βγ}. Further, intracellular perfusion with G_{βγ} enabled D₁ receptor activation to enhance spike firing, while D₂ receptor function was not altered. Finally, our data suggest that these pathways may increase spike firing by inhibition of a slow A-type potassium current. These results provide evidence for a novel cellular mechanism through which cooperative action of D₁ and D₂ receptors in the nucleus accumbens could mediate dopamine-dependent behaviors.

Materials and Methods

Slice Preparation and Electrophysiology.

Coronal slices (300 μm) were prepared from male P22-P28 Sprague-Dawley rats (50-80 g). After cutting, slices recovered at 32° C. in carbogen-bubbled ACSF (126 mM NaCl, 1.6 mM KCl, 1.2 mM NaH₂PO₄, 1.2 mM MgCl₂, 2.4 mM CaCl₂, 18 mM NaHCO₃, 11 mM glucose, with pH 7.2-7.4 and osmolarity 301-305) for 30 minutes to 5 hours. During experiments, slices were submerged and continuously perfused (using a peristaltic pump, ˜2 ml/min) with carbogen-bubbled ACSF warmed to 31-32° C., and supplemented with CNQX (10 μM, to block AMPA-type glutamate miniature EPSPs), picrotoxin (50 μM, to block GABA-A receptors), and sodium metabisulfite (50 μM), an antioxidant to preserve DAergic reagents (Nicola and Malenka, 1997). CNQX and picrotoxin were added to isolate the cell from several major sources of neurotransmitter input whose release is known to be inhibited by dopamine (Pennartz et al., 1992a; Nicola and Malenka, 1997). In preliminary experiments, DA-induced increases in spike firing were observed in the absence of these 3 reagents (data not shown). All reagents were bath applied.

All experiments were performed using whole-cell recording, except where specifically indicated that amphotericin perforated-patch was utilized. Patch-clamping was performed using visualized infrared-DIC with 2.5 to 3.5 MΩ electrodes. Current pulses were applied using Clampex 8.0 and an Axo-1D patch amplifier in current-clamp mode (Axon Instruments, Foster City, Calif.). Upon breaking into neurons, the resting membrane potentials were between −95 and −80 mV. In most experiments, the membrane potential for each neuron was set to ˜−85 mV using the patch amplifier ˜5 minutes after breaking into a cell, except for experiments involving intracellular perfusion with okadaic acid, norokadaone, or G_{βγ} subunits, where cells were held at −90 or −80 mV throughout experiments to compensate for small drifts in membrane potential that can occur after break-in. Series resistance correction was 15-20 MΩ. To record EPSPs, a bipolar stimulating electrode was placed ˜100 μM lateral to the recording electrode. Afferents were stimulated with 10 pulses at 20 Hz (20 μs) every 30 seconds using a Master 8 (AMPI, Jerusalem, Israel), and EPSPs were recorded using Clampex in current-clamp mode.

For voltage-clamp experiments, ACSF was modified to contain 3.6 mM MgCl₂, 0.2 mM CaCl₂, 0.1 mM CdCl₂, and 1 μM TTX to block calcium and sodium current. Neurons were held at −80 mV with the patch amplifier. Our voltage protocol for determining effects of DA and 4-AP on potassium currents involved a series of eleven steps. In each step, a neuron was hyperpolarized to −100 mV for 700 ms, then depolarized for 300 ms. The depolarization ranged from −80 to +20 mV across the eleven steps. This protocol was applied every 30 seconds. For analysis, magnitude of the total evoked potassium current was determined 280 ms into the current response after depolarization to 0 mV.

All data are shown as mean plus or minus the standard error of the mean. Unless otherwise indicated, all statistics were performed using a two-tailed, unpaired t-test.

Analysis of Spike Firing.

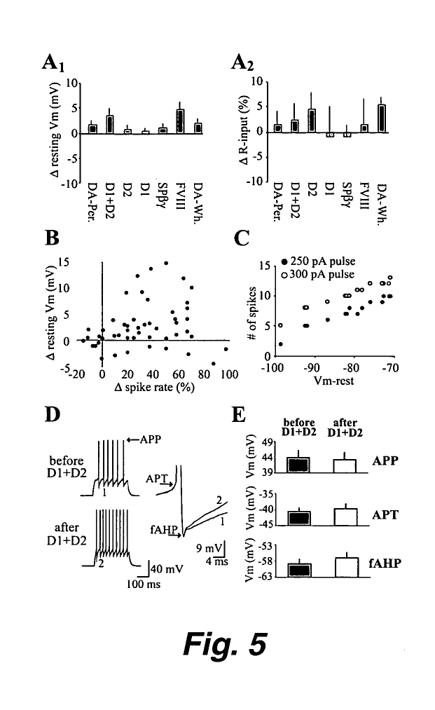

All such data was analyzed using Axograph (Axon Instruments, Foster City, Calif.). To calculate percent change in spiking, a current pulse was selected that exhibited approximately 7-8 spikes at baseline. The same current pulse was used for all time points of a given cell. Spike firing rates during the 3 minutes before addition of the reagent were averaged and this value normalized to 100%. Statistical significance was determined for the average spike firing change during the last 2 minutes of exposure to reagents. In most cases, statistical significance of changes in spike rate for a particular experimental condition was determined in comparison to an appropriate control condition. For experiments where we systematically varied Vm-rest and determined the relationship between change in Vm-rest and change in spike firing, we first determined, for each cell, the change in number of spikes per mV change in Vm-rest. Then, to make this data comparable to the percent change in spike firing measured with DAergic agonists, we chose a baseline number of spikes of 7.2 (the mean number of spikes before addition of DAergic agonists in the cells shown in FIG. 5 C), and determined the percent increase in spike firing per mV change in Vm-rest for each cell.

We attempted to estimate the proportion of neurons that responded to a given treatment, and, as is commonly observed (e.g., Uchimura et al., 1986; Surmeier et al., 1995), we found that not all MSNs responded to DAergic agonists. ˜70% of cells responded under whole-cell conditions, and ˜80% of cells responded under perforated-patch conditions (with a threshold of 15% increase in spiking to be considered responding), suggesting that a small washout of some signaling molecules might have occurred under whole-cell conditions. However, to avoid the arbitrary designation required to delineate cells as responders or not, we included all neurons exposed to a given condition in all of our analyses.

Reagents.

Whole-cell experiments were performed with potassium methanesulfonate- or potassium chloride-based solutions. KMeS: KOH 0.95% (v/v), methanesulfonic acid 0.38% (v/v), 20 mM HEPES, 0.2 mM EGTA, 2.8 mM NaCl, 2.5 mg/ml MgATP, 0.25 mg/ml GTP, pH 7.2-7.4, 275-285 osmolarity; KCl: KCl 144 mM replacing KOH and methanesulfonic acid. Amphotericin experiments used the methanesulfonate-based solution, except without ATP and GTP. Amphotericin was made fresh as a 30 mg/ml stock in DMSO, sonicated, then added at 0.2% (v/v) to pipette solution containing 0.25 mg/ml pluronic, and sonicated again. Peptide sequences were FVIII: YEDSYEDISAYLLSKNNAIPR (SEQ ID NO:1) nd SPβγ: DALRIQMEERFMASNPSKVSYEPIT (SEQ ID NO:2) (Ma et al., 1997, synthesized by Synpep, Dublin, Calif.). Peptides were prepared as a 500× stock in DMSO and kept at −80° C. Peptides were used within 2 weeks of dilution in DMSO. In half of the peptide experiments, the identity of the peptide was not known to the experimenter. Purified bovine whole brain G_{βγ} subunits (Calbiochem, Huang et al., 1998) were aliquoted and kept at −80° C. Maltose binding protein (a generous gift from Dorit Ron and Alicia Vagts), which is approximately the same molecular weight as G_{βγ} (˜50 kD for MBP, ˜46 kD for G_{βγ}), was prepared in the same vehicle as G_{βγ} subunits, with a final concentration of 20 μM DTT, 20 μM EGTA, and 0.002% Lubrol.

Most reagents were prepared fresh each day, including 4-aminopyridine, DA, sodium metabisulfite (all in Ringers), ω-conotoxin-GVIA and ω-agatoxin (in water), eticlopride, NPA, okadaic acid, quinpirole, SCH23390, and U0126 (all in DMSO), and nifedipine (in 95% ethanol, Bargas et al., 1994). Dendrotoxin (Alomone Labs, Jerusalem, Israel), CNQX, Rp-cAMPS (Biolog, La Jolla, Calif.) and amphetamine were dissolved in water and kept at −20° C. SKF81297, SKF82957, forskolin, and dideoxyforskolin were dissolved in DMSO and kept at −20° C. >85% of D1 agonist experiments utilized SKF81297, and, where tested, we saw similar results with both. Picrotoxin was dissolved in water and kept as a room temperature stock. Unless otherwise indicated, all reagents were made at 1:1000 stock, and were purchased from Sigma or RBI.

Results

Co-Activation of D₁ and D₂ Receptors Increases Spike Firing

Coherent excitatory synaptic inputs in vivo drive MSNs from a strongly hyperpolarized state, the “down-state”, to a depolarized state, the “up-state”, which is close to the threshold for action potential generation (Plenz and Kitai, 1998; Wickens and Wilson, 1998; Nicola et al., 2000). Although dopamine can have a number of effects within the basal ganglia (Greengard et al., 1999; Nicola et al., 2000), including modulation of release of several transmitters (McGinty, 1999), we focused upon the postsynaptic effects of dopamine receptor activation on spike firing. Understanding how dopamine could modulate spike firing is critical, since spike firing is a major mechanism by which neurons process information. In addition, there is considerable interest in modulation of spike firing of NAcb MSNs in relation to behavioral events (e.g., see Schultz et al., 1992, Bowman et al., 1996).

We used two criteria to restrict our investigation to MSNs. First, we only recorded from medium-sized cells to exclude the much larger cholinergic interneurons. The majority of neurons showed the slow, repetitive spike firing pattern typically reported for MSNs (Nisenbaum et al., 1994; Plenz and Kitai, 1998; Wickens and Wilson, 1998; Mahon et al., 2000), and all such neurons were included for study. A small proportion of cells (˜5%) exhibited a clearly different firing pattern, with higher rates of firing, a larger fast afterhyperpolarization, and a more depolarized resting membrane potential. These properties are typical of the fast-spiking GABAergic interneurons (Plenz and Kitai, 1998; Bracci et al., 2002), and these cells were not analyzed further.

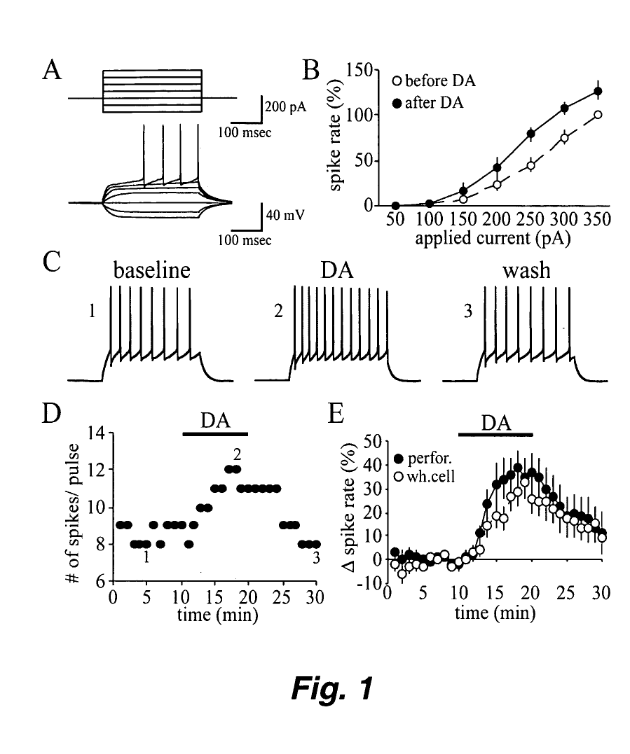

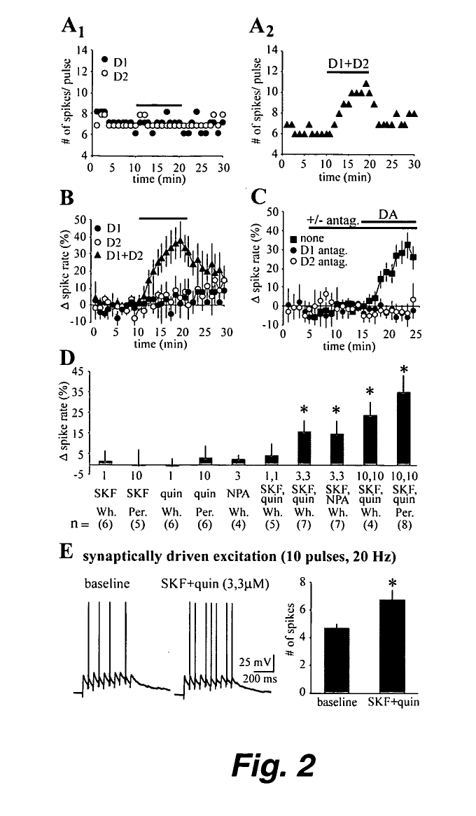

To test the firing properties of MSNs during continuous depolarization, a series of 300 ms current pulses was delivered to a MSN every 30 seconds. The current pulses ranged from −100 pA (hyperpolarizing) to +350 pA (depolarizing, both sub- and supra-threshold for action potentials) in 50 pA steps (FIG. 1 A). Bath application of either 75 or 30 μM DA significantly and reversibly elevated spike firing (75 μM: FIGS. 1 B-E, n=10 and 27 for perforated patch and whole cell experiments, respectively; 30 μM: 17.3+/−6.6%, n=7; both p<0.05, paired t-test), but 10 μM DA did not (1.9+/−4.7%, n=5). Spike firing was also significantly enhanced by amphetamine (10 μM, 18.5+/−5.4%, n=5, p<0.05, paired t-test), which causes release of DA by reversal of the DA transporter (Seiden et al., 1993). However, spike firing was not altered by a selective D₁ agonist alone (SKF 81297 or SKF 82957, 1-10 μM: FIG. 2 A1, B, D) or by a selective D₂ agonist alone (quinpirole, 1-10 μM: FIG. 2 A1, B, D; propylnorapomorphine, NPA, 3 μM: FIG. 2 D). Instead, spike firing was significantly increased after exposure to a D₁ agonist and the D₂ agonist quinpirole in combination with 3 μM of each (FIG. 2 D, p<0.05, paired t-test) or 10 μM of each (FIG. 2 A2, B, D, p<0.05 vs. D₁ or D₂ agonist alone), but not with 1 μM of each (FIG. 2 D). Enhancement of spike firing was also observed using a D₁ agonist in combination with NPA, a D₂ receptor agonist structurally unrelated to quinpirole (FIG. 2 D). Thus, only D₁ and D₂ agonists in combination produced an enhancement in spike firing similar to that observed with DA.

If the DA-mediated increase in spike firing required cooperative activation of D₁ and D₂ receptors, then either a D₁ or a D₂ antagonist should block this activation. DA-mediated enhancement of spike firing was prevented by pre-exposure to either the D₁ antagonist SCH 23990 (1 μM: −0.5+/−4.6%, n=5; 10 μM: FIG. 2 C, n=6; both concentrations p<0.05 vs. DA without antagonists) or the D₂ antagonist eticlopride (300 nM: 2.5+/−5.5%, n=6; 3 μM: FIG. 2 C, n=11; both concentrations p<0.05 vs. DA without antagonists). Therefore, DA-mediated increases in spike firing required activation of both D₁ and D₂ receptors.

To address whether D₁ and D₂ receptor signaling might involve a synaptically released factor, slices were pre-incubated for 15-60 minutes with irreversible antagonists of the N-type (ω-conotoxin GVIA, 500 nM) and P/Q-type (ω-agatoxin IVA, 250 nM) calcium channels, as well as continuous exposure to the L-type calcium channel antagonist nifedipine (30 μM). This treatment completely inhibited evoked glutamatergic EPSCs even one hour after exposure to toxins (data not shown), but did not prevent the enhancement in spike firing by DA (24.0+/−5.0%, n=6, p<0.05, paired t-test). These data suggest that the DA-mediated signaling events did not require a synaptically-released factor.

Since firing of NAcb neurons in vivo usually requires glutamatergic excitation in order to elicit action potentials (Plenz and Kitai, 1998; Wickens and Wilson, 1998; Nicola et al., 2000), we determined whether activation of DA receptors would increase the number of spikes evoked during synaptically-driven spike firing. Thus, using 10 pulses at 20 Hz (with stimulation current set to evoke 4-5 spikes in the basal condition), we found that exposure to a combination of 3 μM each of SKF81297 and quinpirole significantly enhanced the number of spikes elicited by synaptically-driven excitation (FIG. 2 E, from 4.7+/−0.3 spikes to 6.8+/−0.7 spikes, n=5, p<0.05). These results suggest that DA receptor activation can enhance spike firing under conditions that more closely mimic the in vivo situation.

DA-Receptor-Mediated Increase in Spike Firing Requires cAMP and G-Protein βγ Subunits

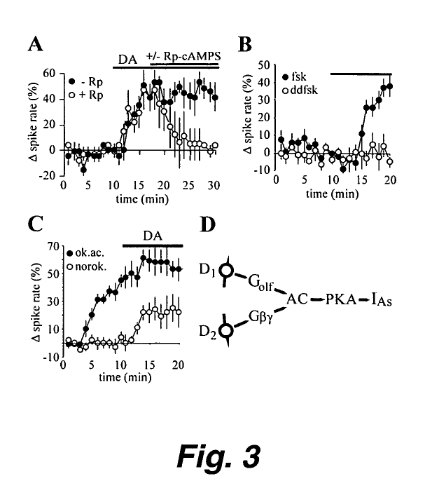

Several studies suggest that PKA plays a major role in DA signaling (Greengard et al., 1999). Addition of 100 μM Rp-cAMPS (Rp), an inhibitor of cAMP-dependent processes, during the DA response significantly reduced spike firing to pre-DA levels (FIG. 3 A, n=4 with Rp, n=5 without Rp, p<0.01). However, Rp alone did not effect basal firing activity (−3.8+/−2.6%, n=4). These data suggest that the cAMP system did not regulate spike rate under basal conditions, but was required for expression of the increased firing rate observed during exposure to DA. In support of this possibility, forskolin (FSK, 5 μM, n=12), an activator of adenylyl cyclases, increased spike firing to a similar degree as DAergic agonists, while dideoxy-forskolin (ddFSK, 5 μM, n=6), an inactive analog of forskolin, had no effect (FIG. 3 B).

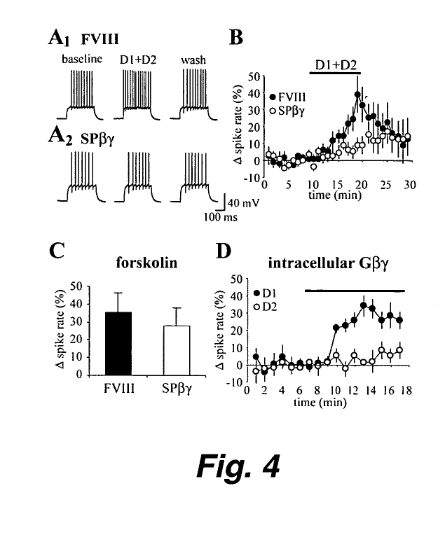

One intracellular mechanism that might underlie cooperativity of D₁ and D₂ receptors is activation of specific subtypes of adenylyl cyclases by G-protein beta-gamma subunits (G_{βγ}) released from the G_{i/o}-linked D₂ receptor in combination with G_{αs}-like subunit signaling from the D₁ receptor. This G_{αs}/G_{βγ} interaction allows G_{i/o}-linked receptors to contribute to, rather than oppose, activation of the protein kinase A (PKA) system (FIG. 3 D) (Sunahara et al., 1996; Watts and Neve, 1997). In this regard, dialysis of neurons with 200 μM of SPβγ (n=9), an inhibitory peptide that interferes with binding of G_{βγ} to several targets (Ma et al., 1997), prevented the increase in spike firing elicited by D₁/D₂ co-activation, while 200 μM of the inactive peptide FVIII (n=10) had no effect (FIG. 4 A, B, p<0.05). Activation of spike firing by forskolin was not prevented by either SPβγ or FVIII (FIG. 4 C, n=4 and n=6, respectively), indicating that the inhibition mediated by SPβγ was upstream of adenylyl cyclase.

If D₁/D₂ receptor cooperativity occurred via the G_{βγ} -dependent mechanism described above, we would predict that, after intracellular perfusion with G_{βγ} subunits, D₁ receptor agonists should enhance spike firing while D₂ receptor agonists should not. Intracellular perfusion with purified bovine brain G_{βγ} subunits (20 nM) had no effect alone (3.7+/−3.6% change in spiking 10 minutes after break-in, n=9). However, a D₁ agonist (SKF81297, 10 μM, n=5) but not a D₂ agonist (quinpirole, 10 μM, n=4) significantly enhanced spike firing in cells perfused with G_{βγ} (FIG. 4 D, p<0.05). The D₁ agonist did not enhance spike firing in neurons perfused with the G_{βγ} vehicle plus maltose binding protein, which is similar in size to G_{βγ} , (−2.5+/−2.5%, n=5). Although we cannot completely rule out effector sites for G_{βγ} other than adenylyl cyclases, taken together these data strongly suggest that G_{βγ} and the cAMP system are required for the D₁/D₂-mediated enhancement of spike firing.

PKA signaling can involve other downstream signaling molecules such as MAP kinase (Impey et al., 1998) and protein phosphatase 1 (PP1) (Surmeier et al., 1995; Schiffmann et al., 1998; Greengard et al., 1999). The MAP kinase inhibitor U0126 (10 μM), which blocks long-term potentiation (LTP) in cortical neurons in slice (Di Cristo et al., 2001), did not prevent DA-induced enhancement of spike firing (22.9+/−5.8%, n=4, p<0.05, paired t-test). However, spike firing was significantly enhanced by intracellular perfusion of okadaic acid (1 μM, FIG. 3 C, n=5, p<0.01, paired t-test), an inhibitor of PP1 and PP2A, and this enhancement occluded DA-mediated changes in spike firing (p>0.1, paired t-test). Norokadaone (1 μM), an inactive analog of okadaic acid, had no effect by itself and did not prevent the effects of DA (FIG. 3 C, n=5). In several studies, okadaic acid mimics the inhibition of PP1 by DARPP-32 (Surmeier et al., 1995; Schiffmann et al., 1998), which is normally PKA-dependent (Greengard et al., 1999), suggesting that the DA- and PKA-mediated enhancement of spike firing observed here may involve signaling through DARPP-32.

Role of Slow A-type Potassium Current in Spike Firing Enhancement

A number of ionic mechanisms have been reported to be modulated by dopamine (for review, see Greengard et al., 1999; Nicola et al., 2000). Therefore, we first analyzed several baseline electrophysiological parameters that might be altered upon exposure to DA or the combination of a D₁ and a D₂ receptor agonist. However, we observed no significant changes in resting membrane potential (FIG. 5 A1, Vm-rest) or input resistance (FIG. 5 A2, R-input, both p>0.25, each parameter tested by one way ANOVA across all groups shown in FIG. 5 A1). We also performed a within-cell comparison of the spike firing change and the change in input resistance or Vm-rest. For this analysis, we grouped cells exposed to 75 μM DA and 10 μM each of the D₁ and D₂ receptor agonists, n=51 cells). Although change in R-input did not significantly correlate with change in spike firing (p>0.2, data not shown, Spearman rank order correlation), the change in Vm-rest nearly did (p>0.07, FIG. 5 B). To further address this issue, we systematically varied Vm-rest, and determined the relationship between change in Vm-rest and change in spike firing (3.5+/−0.4% increase in spike firing per mV depolarization, n=5, FIG. 5 C, see Materials and Methods for details of analysis). Thus, for the majority of neurons shown in FIG. 5 B, the contribution of the change in Vm-rest to change in spike firing is negligible. Also, in several experiments (intracellular perfusion with G_{βγ}, okadaic acid, or norokadaone), we clamped the cell at −80 or −90 mV throughout the experiment, and still observed enhancement of spike firing with dopaminergic agonists. Thus, a mechanism independent of changes in Vm-rest primarily accounts for the elevation in spike firing observed here, although DA-dependent depolarization can occur in a minority of cells.

We also measured a number of features of the action potential, and found that DAergic activation did not significantly change action potential threshold (APT), width (APW), and peak (APP), fast afterhyperpolarization (fAHP), and magnitude of depolarization by sub-threshold current pulses (data not shown) (all p>0.25, each parameter tested by one way ANOVA across all groups shown in FIG. 5 Al). FIG. 5 D shows an example cell with an overlay of the action potential before and after exposure to D₁ and D₂ agonists in combination, while averaged data for APP, APW, and fAHP are shown in FIG. 5 E. In particular, several changes that would be predicted by alterations in the function of sodium channels (changes in APT, APW, and APP; Calabresi et al., 1987; Schiffmann et al., 1998) and the delayed rectifier potassium channel (changes in APW and fAHP, Rudy and McBain, 2001) were not observed (FIG. 5 D, E).

Thus, DA or the combination of a D₁ and a D₂ receptor agonist enhanced spike firing without altering baseline parameters or several features of the action potential. Changes in calcium channel function (Surmeier et al., 1995; Cepeda et al., 1998) might underlie the observed pattern, but the experiments described above using calcium channel antagonists suggested that L-, N-, and P/Q-type calcium channels were not required for the DA-related enhancement of spike firing. Instead, our data indicate that the DAergic enhancement of spike firing was mediated by inhibition of I_{As} (Surmeier et al., 1991; Surmeier and Kitai, 1993; Nisenbaum et al., 1994; Gabel and Nisenbaum, 1998; Mahon et al., 2000).

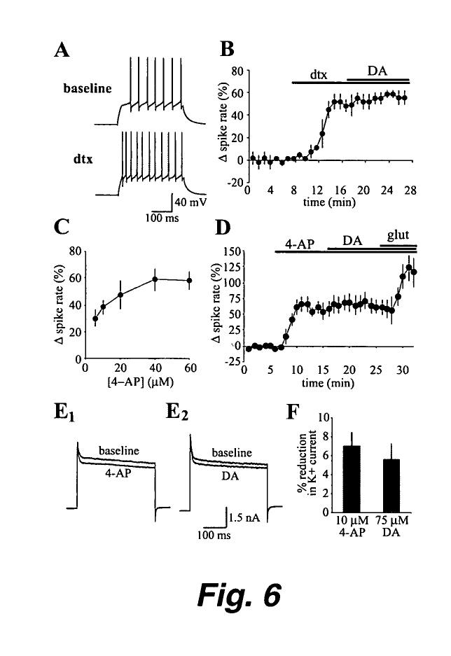

We tested pharmacological inhibitors of I_{As}, including α-dendrotoxin (α-dtx), which is highly selective for I_{As}, and 4-aminopyridine (4-AP), which is relatively selective for I_{As} at a concentration range of 5-60 μM (Surmeier et al., 1991; Nisenbaum et al., 1994). All these compounds significantly enhanced spike firing (α-dtx: 0.5 μM, FIG. 6 A, B, n=4; 4-AP: 5-60 μM, FIG. 6 C, D; all p<0.01, paired t-test). The enhancement of firing observed with 10 μM 4-AP persisted in the presence of the combination of calcium channel inhibitors described above (25.7+/−4.2%, n=4), suggesting an action via a postsynaptic mechanism. Further, (α-dtx and 4-AP significantly occluded the effects of DA (α-dtx: FIG. 6 B, n=4; 10 μM 4-AP: 11.4+/−5.1%, n=5; 60 μM 4-AP: FIG. 6 D, n=6; all p>0.05, paired t-test testing the effect of DA). Occlusion was not simply due to a limitation on the number of spikes a neuron could fire after exposure to 60 μM 4-AP, as application of glutamate (200 μM) further increased spike rate (FIG. 6 D, p<0.01, paired t-test). In addition, the enhancement of spike firing by okadaic acid was occluded by ˜13 minutes pre-exposure to 60 μM 4-AP (16+/−4.3% firing change with okadaic acid in the presence of 4-AP, n=4, p<0.05, vs. okadaic acid without pre-exposure to 4-AP). Taken together, these occlusion experiments suggest that DA, G_{βγ}, okadaic acid, α-dtx, and 4-AP all enhance spike firing by a common mechanism, inhibition of I_{As}.

We also examined the effects of DA and 4-AP on total potassium currents, comprised of I_{As}, delayed-rectifier, and non-inactivating potassium currents (Surmeier et al., 1991; Surmeier and Kitai, 1993; Nisenbaum et al., 1994; Gabel and Nisenbaum, 1998; Mahon et al., 2000; Rudy and McBain, 2001), by performing voltage-clamp experiments where sodium and calcium currents were blocked (for details, see Materials and Methods). DA (75 μM, n=8) and 4-AP (10 μM, n=6) produced a similar small but significant inhibition in the potassium current evoked by a 300 ms pulse to 0 mV (FIG. 6 E-F; DA and 4-AP both p<0.05 change in current, paired t-test), consistent with previous reports showing that I_{As} contributes a minor amount to the total evoked potassium current (Surmeier and Kitai, 1993; Bekkers and Delaney, 2001). However, Bekkers and Delaney (2001) showed that, despite the modest contribution of I_{As} to the total potassium current, inhibition of I_{As} produces a significant enhancement in spike firing, emphasizing the critical role that I_{As} plays in regulation of action potential firing (Nisenbaum et al., 1994; Wickens and Wilson, 1998; Mahon et al., 2000).

Current-clamp experiments of I_{As} in striatal MSNs also suggest that I_{As} is a key regulator of the latency to firing the first action potential during prolonged depolarization (e.g., see FIG. 1 A) (Nisenbaum et al., 1994; Mahon et al., 2000), and thus any condition that inhibits I_{As} should shorten the latency to firing. As shown in Table 1, a significant reduction in latency to fire was observed after exposure to DAergic agonists, forskolin, or antagonists of I_{As}. These data further support the contention that DA, okadaic acid, α-dendrotoxin, and 4-AP enhanced spike firing through inhibition of I_{As}.

Discussion

This example shows that DA increased spike firing in MSNs from the NAcb shell. This enhancement of spike firing required co-activation of D₁ and D₂ receptors, as neither agonist alone modified spike firing, and the effect of DA was inhibited by either a D₁ or a D₂ receptor antagonist. The increased spike firing after co-activation of D₁ and D₂ receptors was mediated intracellularly by a mechanism requiring activation of G_{βγ} and cAMP-dependent processes. Finally, our biophysical and pharmacological studies suggested that enhancement of spike firing occurred through inhibition of a slow A-type potassium current.

Our results may provide a cellular mechanism to explain observations from behavioral studies suggesting a cooperative action of D₁ and D₂ receptors in the NAcb. For example, rats will self-administer D₁ and D₂ agonists into the NAcb in combination, but will not self-administer either alone (Ikemoto et al., 1997). Both synergistic and additive effects of D₁ and D₂ receptor activation in the NAcb have been reported by studies of conditioned reinforcement (Chu and Kelley, 1992; Wolterink et al., 1993). Also, several studies have observed D₁/D₂ cooperativity during locomotor activation, although higher doses of D₁ or D₂ agonist alone can enhance locomotion (Plaznik et al., 1989; Gong et al., 1999). Finally, results from studies of amphetamine (Phillips et al., 1994) and ethanol (Hodge et al., 1997) self-administration and evaluating the relative cost of obtaining a reward (Koch et al., 2000; Nowend et al., 2001) are also suggestive of a cooperative role for D₁ and D₂ receptors in the NAcb in behavioral expression. Although D₁/D₂ interaction is not observed for all behaviors that require DA in the NAcb (e.g., Coccurello et al., 2000), these studies suggest that D₁ and D₂ receptors interact cooperatively in the expression of a number of reward- and motivation-related behaviors mediated by the NAcb.

The observation that D₁ and D₂ receptors may act cooperatively in the NAcb during expression of some behaviors is quite intriguing, given that D₁ and D₂ receptors are traditionally thought to oppositely couple to the G-protein/PKA system (Missale et al., 1998). G_{βγ} provides a mechanism by which Gₛ- and G_{i/o}-coupled receptors, such as D₁ and D₂, respectively, can act cooperatively to activate PKA (Sunahara et al., 1996, Watts and Neve, 1997), especially perhaps for behaviors involving NAcb PKA signaling (Self et al., 1998; Sutton et al., 2000). A key role for G_{βγ} in behavior was demonstrated in a recent paper finding that self-administration of ethanol is significantly reduced after inhibition of G_{βγ} function in the NAcb (Yao et al., 2002), in agreement with previous studies showing decreased ethanol consumption after block of D₁ or D₂ receptors within the NAcb (Hodge et al., 1997). Here, enhancement of spike firing after co-activation of D₁ and D₂ receptors required both G_{βγ} and cAMP-dependent processes. In particular, intracellular perfusion with G_{βγ} enabled D₁ but not D₂ enhancement of spike firing, indicating that G_{βγ} derived from D₂ was required for spike firing increases. These data raise the interesting possibility that the DAergic signaling pathway we have identified mediates self-administration of ethanol and perhaps other behaviors.

Our results are also consistent with studies suggesting that PKA plays a major role in DA signaling in MSNs (Greengard et al., 1998). Also, several adenylyl cyclase isoforms sensitive to G_{βγ}-dependent activation (Sunahara et al., 1996) are present in the NAcb (Hellevuo et al., 1996; Mons et al., 1998). We should note that there are likely to be multiple forms of DA receptor and G_{βγ} signaling, including presynaptic modulation (McGinty, 1999) and interaction with signaling pathways other than PKA (Seiden et al., 1993; Sunahara et al., 1996; Missale et al., 1998; Hernandez-Lopez et al., 2000). Also, although we used relatively high concentrations of DA (see also Pennartz et al., 1992a; Nicola and Malenka, 1997), the high density of dopamine transporters around MSNs (Uchimura and North, 1990; Jones et al., 1995; Hersch et al., 1997) make it likely that the very strong dopamine transporter activity present in slice (Uchimura and North, 1990; Jones et al., 1995) greatly reduces the extracellular concentration of DA.

Our results with inhibitors of calcium channels suggest that D₁/D₂ signaling does not require synaptic transmission, raising the possibility that D₁ and D₂ receptors are co-localized to the same cell. However, previous studies have reported a diversity of estimates of the degree of D₁ and D₂ receptor co-localization among MSNs, which may reflect varying sensitivity of different methodologies (for review, see Aizman et al., 2000; Nicola et al., 2000). Here, we provide a novel mechanism by which D₁ and D₂ receptors can act in cooperation to enhance spike firing, although we cannot definitively address whether D₁ and D₂ receptors are localized to the same or different neurons. If the receptors are localized to different neurons, our results using intracellular perfusion with G_{βγ} suggest that D₂ receptors are localized to the neuron being recorded from, as intracellular perfusion with G_{βγ} mimics D₂ receptor input and enables D₁ receptor activation (which could come from the same or a different cell) but not D₂ activation. Also, the results we obtained with calcium channel antagonists suggest that any between-cell communication will not be mediated by a synaptically-released factor.

Our data also indicate that DA, via interaction between D₁ and D₂ receptors, might increase the firing rate of MSNs in the NAcb in vivo. However, studies of DAergic modulation of MSN spike firing have produced mixed results both in vivo and in vitro, with observations of both excitation and inhibition (for review, see Siggins, 1978; Nicola et al., 2000). Several factors might contribute to these discrepancies. First, DAergic reduction of firing might be due to inhibition of glutamate release (Pennartz et al., 1992a; Nicola et al., 1996; Nicola and Malenka, 1997). Second, several studies have observed dose-dependent effects of DA, with lower doses activating and higher doses inhibiting firing (Chiodo and Berger, 1986; Wachtel et al., 1989; Williams and Millar, 1990; Hu and White, 1997). In this regard, DA release after stimulation of the VTA or the median forebrain bundle, which might result in more moderate DA levels compared to direct application, can strongly enhance spike firing in MSNs (Chiodo and Berger, 1986; Gonon and Sundstrom, 1996). Thus, DA likely has multiple effects, perhaps depending on dose or signaling context, but there is a strong precedent for DAergic activation of MSNs. Of particular interest are recent studies of NAcb firing in response to cues that indicate food reward. In some NAcb cells, firing rates increase during presentation of the cue, and this enhancement of firing is greatly reduced by VTA inactivation. VTA inactivation or infusion of dopamine receptor antagonists into the NAcb also greatly inhibits behavioral responding to the cue. Taken together, these data suggest that DA enhances firing in a set of NAcb neurons, and that this change in firing may be important for proper task performance after the cue is observed.

Several factors may also contribute to apparent contradictions among in vitro studies. Some studies from NAcb slice found no DA-related changes in post-synaptic properties (Pennartz et al., 1992a; Nicola et al., 1996; Nicola and Malenka, 1997), while others observed significant DA-dependent changes in input resistance or resting membrane potential (Uchimura et al., 1986; Uchimura and North, 1990; O'Donnell and Grace, 1996). Changes in baseline properties are likely due to action of DA on cell types other than MSNs (Yasumoto et al., 2002), and such influences were negated here by studying DAergic signaling in relative pharmacological isolation from other cells. Some discrepancies may also relate to differences among MSNs from dorsal striatum, NAcb core, and NAcb shell (Kelly and Nahorski, 1987; Calabresi et al., 1992; Pennartz et al., 1992b; O'Donnell and Grace, 1993; Paxinos, 1995; Thomas et al., 2000). In particular, D₁ and D₂ receptors in the dorsal striatum are more clearly segregated in the so-called “patch” and “matrix” compartments, while such distinction is much less clear in the NAcb shell (Paxinos, 1995). As slice studies from the dorsal striatum have generally not addressed the compartmental localization of the neurons under investigation, differential signaling among compartments could also contribute significantly to the variety of DAergic effects observed among in vitro studies from the dorsal striatum (see Nicola et al., 2000, for review).

Pharmacological and biophysical analyses suggest that the DA-mediated enhancement of spike firing observed here was mediated by inhibition of I_{As}, and was not associated with a change in function of several other channel types, including sodium and L-, N-, and P/Q-type calcium channels. Pharmacological inhibitors of I_{As}, such as α-dendrotoxin or 4-AP (5-60 μM), enhanced spike firing and occluded the effects of DA on spike firing. Occlusion of DAergic effects after inhibition of as function strongly suggests that DA enhances spike firing by inhibiting I_{As}. In addition, 60 μM 4-AP occluded the okadaic acid-mediated enhancement of spike firing. Finally, using voltage-clamp methods, we observed a small but significant inhibition of potassium currents by 4-AP and DA (see also Surmeier and Kitai, 1993). In this regard, Bekkers and Delaney (2001) found that inhibition of I_{As} produced only a small decrease in total potassium current, but led to a significant enhancement in spike firing, consistent with the critical role I_{As} contributes to action potential firing firing (Nisenbaum et al., 1994; Wickens and Wilson, 1998; Mahon et al., 2000). Taken together, these data support the suggestion that DA, G_{βγ}, okadaic acid, α-dtx, and 4-AP all enhanced spike firing via inhibition of I_{As}.

Although in the present study we did not observe changes in these parameters, we should note that other groups have observed modulation of sodium channels by DA in the NAcb⁶³.

Based on analyses of the “up-” and “down-state” transitions and biophysical properties, it has been suggested that potassium channels are key regulators of excitability of MSNs (Wickens and Wilson, 1998). I_{As} activates at voltages around spike threshold, and inhibition of I_{As} may allow previously sub-threshold synaptic input to elicit action potential firing (Nisenbaum et al., 1994; Wickens and Wilson, 1998; Mahon et al., 2000). In agreement, we found that DAergic activation increased the number of spikes fired during synaptic stimulation, and also that inhibition of I_{As} with DAergic agonists or direct I_{As} antagonists decreased the latency to firing. Our data predict that DA will enhance the number of spikes fired in vivo during an up-state transition or during any other coherent glutamate excitation, with little effect on the hyperpolarized down-state. This is consistent with the idea that DA is modulatory, and normally requires glutamate receptor activation for DAergic effects to be observed (Chiodo and Berger, 1986; Nicola et al., 2000). It is also interesting that low concentrations of 4-AP mimic the effect of DA, raising the possibility that even moderate inhibition of IAS might produce significant changes in spike rate (see also Bekkers and Delaney, 2001). Thus, by inhibition of I_{As}, co-activation of D₁ and D₂ receptors in the NAcb shell could enhance glutamate-mediated cellular excitation and thereby contribute to the expression of goal-directed behaviors.

References

Aizman O, Brismar H, Uhlen P, Zettergren E, Levey A I, Forssberg H, Greengard P, Aperia A (2000) Anatomical and physiological evidence for D₁ and D₂ dopamine receptor colocalization in neostriatal neurons. Nat Neurosci 3:226-230.

Bargas J, Howe A, Eberwine J, Cao Y, Surmeier D J (1994) Cellular and molecular characterization of Ca²⁺ currents in acutely isolated, adult rat neostriatal neurons. J Neurosci 14:6667-6686.

Bekkers J M, Delaney A J (2001) Modulation of excitability by alpha-dendrotoxin-sensitive potassium channels in neocortical pyramidal neurons. J Neurosci 21:6553-6560.

Bowman E M, Aigner T G, Richmond B J (1996) Neural signals in the monkey ventral striatum related to motivation for juice and cocaine rewards. J Neurophysiol 75:1061-1073.

Bracci E, Centonze D, Bernardi G, Calabresi P (2002) Dopamine excites fast-spiking interneurons in the striatum. J Neurophysiol 87:2190-2194.

Calabresi P, Maj R, Pisani A, Mercuri N B, Bernardi G (1992) Long-term synaptic depression in the striatum: physiological and pharmacological characterization. J Neurosci 12:4224-4233.

Calabresi P, Mercuri N, Stanzione P, Stefani A, Bernardi G (1987) Intracellular studies on the dopamine-induced firing inhibition of neostriatal neurons in vitro: evidence for D1 receptor involvement. Neuroscience 20:757-771.

Cepeda C, Colwell C S, Itri J N, Chandler S H, Levine M S (1998) Dopaminergic modulation of NMDA-induced whole cell currents in neostriatal neurons in slices: contribution of calcium conductances. J Neurophys 79:82-94.

Chiodo L A, Berger T W (1986) Interactions between dopamine and amino acid-induced excitation and inhibition in the striatum. Brain Res 375:198-203.

Chu B, Kelley A E (1992) Potentiation of reward-related responding by psychostimulant infusion into nucleus accumbens: Role of dopamine receptor subtypes. Psychobiology 20:153-162.

Coccurello R, Adriani W, Oliverio A, Mele A (2000) Effect of intra-accumbens dopamine receptor agents on reactivity to spatial and non-spatial changes in mice. Psychopharmacology 152:189-199.

Di Cristo G, Berardi N Cancedda L, Pizzorusso T, Putignano E, Ratto G M, Maffei L (2001) Requirement of ERK activation for visual cortical plasticity. Science 292:2337-2340.

Gabel L A Nisenbaum E S (1998) Biophysical characterization and functional consequences of a slowly inactivating potassium current in neostriatal neurons. J Neurophys 79:1989-2002.

Gong W, Neill D B, Lynn M, Justice J J (1999) Dopamine D₁/D₂ agonists injected into nucleus accumbens and ventral pallidum differentially affect locomotor activity depending on site. Neuroscience 93:1349-1358.

Gonon F, Sundstrom L (1996) Excitatory effects of dopamine released by impulse flow in the rat nucleus accumbens in vivo. Neuroscience 75:13-18.

Greengard P, Allen P B, Nairn A C (1999) Beyond the dopamine receptor: the DARPP-32/protein phosphatase-1 cascade. Neuron 23:435-447.

Hellevuo K, Hoffman P L, Tabakoff B (1996) Adenylyl cyclases: mRNA and characteristics of enzyme activity in three areas of brain. J Neurochem 67:177-185.

Hernandez-Lopez S, Tkatch T, Perez-Garci E, Galarraga E, Bargas J, Hamm H, Surmeier D J (2000) D₂ dopamine receptors in striatal medium spiny neurons reduce L-type Ca2+ currents and excitability via a novel PLCβ1-IP₃-calcineurin-signaling cascade. J Neurosci 20:8987-8995.

Hersch S M, Yi H, Heilman C J, Edwards R H, Levey A I (1997) Subcellular localization and molecular topology of the dopamine transporter in the striatum and substantia nigra. J Comp Neurol 388:211-227.

Hodge C W, Samson H H, Chappelle A M (1997) Alcohol self-administration: further examination of the role of dopamine receptors in the nucleus accumbens. Alc Clin Exp Res 21:1083-1091.

Hu X T, White F J (1997) Dopamine enhances glutamate-induced excitation of rat striatal neurons by cooperative activation of D1 and D2 class receptors. Neurosci Lett 224:61-65.

Huang C L, Feng S, Hilgemann D W (1998) Direct activation of inward rectifier potassium channels by PIP2 and its stabilization by Gbetagamma. Nature 391:803-806.

Ikemoto S, Glazier B S, Murphy J M McBride W J (1997) Role of dopamine D1 and D2 receptors in the nucleus accumbens in mediating reward. J Neurosci 17:8580-8587.

Impey S, Obrietan K, Wong S T, Poser S, Yano S, Wayman G, Deloulme J C, Chan G, Storm D R (1998) Cross talk between ERK and PKA is required for Ca2+ stimulation of CREB-dependent and ERK nuclear translocation. Neuron 21:869-883.

Jones S R, Garris P A, Kilts C D, Wightman R M (1995) Comparison of dopamine uptake in the basolateral amygdaloid nucleus, caudate-putamen, and nucleus accumbens of the rat. J Neurochem 64:2581-2589.

Kelly E, Nahorski S R (1987) Dopamine D-2 receptors inhibit D-1 stimulated cyclic AMP accumulation in striatum but not limbic forebrain. Naunyn-Schmiedebergs Arch Pharm 335:508-512.

Koch M, Schmid A., Schnitzler H U (2000) Role of nucleus accumbens dopamine D₁ and D₂ receptors in instrumental and Pavlovian paradigms of conditioned reward. Psychopharmacology 152:67-73.

LaHoste G J, Henry B L, Marshall J F (2000) Dopamine D₁ receptors synergize with D₂, but not D₃ or D₄, receptors in the striatum without the involvement of action potentials. J Neurosci 20:6666-6671.

Ma J Y, Catterall W A, Scheuer T (1997) Persistent sodium currents through brain sodium channels induced by G protein βγ subunits. Neuron 19:443-452.

Mahon S, Delord B, Deniau J M, Charpier S (2000) Intrinsic properties of rat striatal output neurones and time-dependent facilitation of cortical inputs in vivo. J Physiol (Lond) 527.2:345-354.

McGinty J F (1999) Regulation of neurotransmitter interactions in the ventral striatum. Ann NY Acad Sci 877:129-139.

Missale C, Nash S R, Robinson S W, Jaber M, Caron M G (1998) Dopamine receptors: from structure to function. Physiol Rev 78:189-225.

Mons N, Yoshimura M, Ikeda H, Hoffman P L, Tabakoff B (1998) Immunological assessment of the distribution of type VII adenylyl cyclase in brain. Brain Res 788:251-261.

Nicola S M, Kombian S B, Malenka R C (1996) Psychostimulants depress excitatory synaptic transmission in the nucleus accumbens via presynaptic D1-like dopamine receptors. J Neurosci 16:1591-1604.

Nicola S M, Malenka R C (1997) Dopamine depresses excitatory and inhibitory synaptic transmission by distinct mechanisms in the nucleus accumbens. J Neurosci 17:5697-5710.

Nicola S M, Surmeier D J, Malenka R C (2000) Dopaminergic modulation of neuronal excitability in the striatum and nucleus accumbens. Ann Rev Neurosci 23:185-215.

Nisenbaum E S, Xu Z C, Wilson C J (1994) Contribution of a slowly inactivating potassium current to the transition to firing of neostriatal spiny projection neurons. J Neurophys 71:1174-1189.

Nowend K L, Arizzi M, Carlson B B, Salamone J D (2001) D1 or D2 antagonism in nucleus accumbens core or dorsomedial shell suppresses lever pressing for food but leads to compensatory increases in chow consumption. Pharmacol Biochem Behav 69:373-382.

O'Donnell P, Grace A A (1993) Physiological and morphological properties of accumbens core and shell neurons recorded in vitro. Synapse 13:135-160.

O'Donnell P, Grace A A (1996) Dopaminergic reduction of excitability in nucleus accumbens neurons recorded in vitro. Neuropsychopharm 15:87-97.

Paxinos G (1995) The Rat Nervous System Atlas. New York: Academic Press.

Pennartz C M, Dolleman-Van der Weel M J, Kitai S T, Lopes d S (1992a) Presynaptic dopamine D1 receptors attenuate excitatory and inhibitory limbic inputs to the shell region of the rat nucleus accumbens studied in vitro. J Neurophys 67:1325-1334.

Pennartz C M, Dolleman-Van der Weel M J, Lopes d S (1992b) Differential membrane properties and dopamine effects in the shell and core of the rat nucleus accumbens studied in vitro. Neurosci Lett 136:109-112.

Phillips G D, Robbins T W, Everitt B J (1994) Bilateral intra-accumbens self-administration of d-amphetamine: antagonism with intra-accumbens SCH-23390 and sulpiride. Psychopharmacology 114:477-485.

Plaznik A, Stefanski R, Kostowski W (1989) Interaction between accumbens D1 and D2 receptors regulating rat locomotor activity. Psychopharmacology 99:558-562.

Plenz D, Kitai S T (1998) Up and down states in striatal medium spiny neurons simultaneously recorded with spontaneous activity in fast-spiking interneurons studied in cortex-striatum-substantia nigra organotypic cultures. J Neurosci 18:266-283.

Rudy B, McBain C J (2001) Kv3 channelzs: voltage-gated K+ channels designed for high-frequency repetitive firing. Trends Neurosci 24:517-526.

Schiffmann S N, Desdouits F, Menu R, Greengard P, Vincent J D, Vanderhaeghen J J, Girault J A (1998) Modulation of the voltage-gated sodium current in rat striatal neurons by DARPP-32, an inhibitor of protein phosphatase. Eur J Neurosci 10:1312-1320.

Schultz W, Apicella P, Scarnati E, Ljungberg T (1992) Neuronal activity in monkey ventral striatum related to the expectation of reward. J Neurosci 12:4595-4610.

Seiden L S, Sabol K E, Ricaurte G A (1993) Amphetamine: effects on catecholamine systems behavior. Annu Rev Pharmacol Toxicol 33:639-677.

Self D W, Genova L M, Hope B T, Barnhart W J, Spencer J J, Nestler E J (1998) Involvement of cAMP-dependent protein kinase in the nucleus accumbens in cocaine self-administration and relapse of cocaine-seeking behavior. J Neurosci 18:1848-1859.

Siggins G R (1978) Electrophysiological Role of Dopamine in Striatum: Excitatory or Inhibitory? In: Psychopharmacology: A Generation of Progress (Lipton M A, DiMascio A, Killam K F, eds.), pp. 143-157. New York: Raven Press.

Spanagel R, Weiss F (1999) The dopamine hypothesis of reward: past and current status. Trends Neurosci 22:521-527.

Stoof J C, Kebabian J W (1981) Opposing roles for D1 and D2 dopamine receptors in efflux of cyclic AMP from rat neostriatum. Nature 294:366-368.

Sutton M A, McGibney K, Beninger R J (2000) Conditioned locomotion in rats following amphetamine infusion into the nucleus accumbens: blockade by coincident inhibition of protein kinase A. Behav Pharmacol 11:365-376.

Sunahara R K, Dessauer C W, Gilman A G (1996) Complexity and diversity of mammalian adenylyl cyclases. Ann Rev Pharm Tox 36:461-480.

Surmeier D J, Bargas J, Hemmings H J, Nairn A C, Greengard P (1995) Modulation of calcium currents by a D₁ dopaminergic protein kinase/phosphatase cascade in rat neostriatal neurons. Neuron 14:385-397.

Surmeier D J, Stefani A, Foehring R C, Kitai S T (1991) Developmental regulation of a slowly-inactivating potassium conductance in rat neostriatal neurons. Neurosci Lett 122:41-46.

Surmeier D J, Kitai S T (1993) D₁ and D₂ dopamine receptor modulation of sodium and potassium currents in rat neostriatal neurons. Prog Brain Res 99:309-324.

Thomas M J, Malenka R C, Bonci A (2000) Modulation of long-term depression by dopamine in the mesolimbic system. J Neurosci 20:5581-5586.

Uchimura N, Higashi H, Nishi S (1986) Hyperpolarizing and depolarizing actions of dopamine via D-1 and D-2 receptors on nucleus accumbens neurons. Brain Res 375:368-372.

Uchimura N, North R A (1990) Actions of cocaine on rat nucleus accumbens neurones in vitro. Brit J Pharm 99:736-740.

Watts V J, Neve K A (1997) Activation of type II adenylate cyclase by D2 and D4 but not D3 dopamine receptors. Mol Pharmacol 52:181-186.

Wachtel S R, Hu X T, Galloway M P, White F J (1989) D1 dopamine receptor stimulation enables the postsynaptic, but not autoreceptor effects of D2 dopamine agonists in nigrostriatal and mesoaccumbens dopamine systems. Synapse 4:327-346.

Wickens J R, Wilson C J (1998) Regulation of action-potential firing in spiny neurons of the rat neostriatum in vivo. J Neurophys 79:2358-2364.

Williams G V, Millar J (1990) Concentration-dependent actions of stimulated dopamine release on neuronal activity in rat striatum. Neuroscience 39:1-16.

Wolterink G, Phillips G, Cador M, Donselaar-Wolterink I, Robbins T W, Everitt B J (1993) Relative roles of ventral striatal D₁ and D₂ dopamine receptors in responding with conditioned reinforcement, Psychopharmacology 110:355-364.

Yao L, Arolfo M P, Dohrman D P, Jiang Z, Fan P, Fuchs S, Janak P H, Gordon A S, Diamond I (2002) Betagamma dimers mediate synergy of dopamine D2 and adenosine A2 receptor-stimulated PKA signaling and regulate ethanol consumption. Cell 109:733-743.

Yasumoto S, Tanaka E, Hattori G, Maeda H, Higashi H (2002) Direct and Indirect Actions of Dopamine on the Membrane Potential in Medium Spiny Neurons of the Mouse Neostriatum. J Neurophysiol 87:1234-1243.

Zahm D S (1999) Functional-anatomical implications of the nucleus accumbens core and shell subterritories. Ann. NY Acad Sci 877:113-128.

Zhang X F, Hu X T, White F J (1998) Whole-cell plasticity in cocaine withdrawal: reduced sodium currents in nucleus accumbens neurons. J Neurosci 18:488-498.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening for an agent that modulates self-administration of a substance of abuse, said method comprising:
contacting a neural cell with a test agent; and
determining whether said test agent agonizes activity of a slow A-type potassium current (I_{AS}), wherein an increase in the activity of said potassium current indicates that said test agent is an agent that is expected to inhibit self-administration of a substance of abuse.

2. The method of claim 1, wherein said determining comprises an electrophysiological measurement.

3. The method of claim 1, wherein said neural cell is in a brain tissue.

4. The method of claim 1, wherein said neural cell is in a brain slice preparation.

5. The method of claim 4, wherein said brain slice preparation comprises tissue of the nucleus accumbens.

6. The method of claim 1, wherein said neural cell is a nucleus accumbens cell.

7. The method of claim 1, wherein said test agent is a small organic molecule.

8. A method of inhibiting nucleus accumbens spike firing in response to administration of a substance of abuse, said method comprising increasing activity of a slow A-type potassium current (I_{AS}) in cells of the nucleus accumbens.

9. The method of claim 8, wherein said substance of abuse is selected fro the group consisting of ethanol, an opiate, a cannabinoid, a stimulant, and nicotine.

10. The method of claim 8, wherein said inhibiting comprises administering a small organic molecule that inhibits activity of said slow A-type potassium current.

11. A method of inhibiting self-administration of a substance of abuse, said method comprising increasing activity of a slow A-type potassium current (I_{AS}).

12. The method of claim 11, wherein said substance of abuse is selected fro the group consisting of ethanol, an opiate, a cannabinoid, a stimulant, and nicotine.

13. The method of claim 12, wherein said substance of abuse is alcohol

14. The method of claim 11, wherein said inhibiting comprises administering a small organic molecule that inhibits activity of said slow A-type potassium current.

15. The method of claim 11, wherein said inhibiting comprises electrophysiologically inhibiting said slow A-type potassium current.

16. A composition for mitigating symptoms of consumption or withdrawal of a substance of abuse, said composition comprising a modulator of a slow A-type potassium current.

17. The composition of claim 16, wherein said composition further comprises a pharmacologically acceptable excipient.
